(19) United States
(12) Patent Application Publication
(10) Pub. No.: US 2010/0114310 A1
(43) Pub. Date: May 6, 2010

(54) Breast Prosthesis and Breast Augmentation Using the Breast Prosthesis

(75) Inventor: Nobuyuki Masaki, Tokyo (JP)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Nobuyuki MASAKI c/o: Masaki Clinic, Daikanyama Parkside Village, Shibuya-ku (JP)

(21) Appl. No.: 12/443,127

(22) PCT Filed: Oct. 24, 2006
(86) PCT No.: PCT/JP2007/066567

§ 371 Date: Mar. 26, 2009

(30) Foreign Application Priority Data
Oct. 24, 2006 (JP) 2006-288953

(51) Int. Cl. A61F 2/12

(52) U.S. Cl. 623/8

(57) ABSTRACT

An outer bag in which a core is contained in a movable state is made of a non-assimilatable material to function as an outer artificial coating. The outer bag includes a sheet-shaped base and a large number of convexes (14, 15) and/or concaves 14 formed integrally with the sheet-shaped base and disposed on an outer surface of the sheet-shaped base. Therefore, there is no probability that a breast prosthesis implanted into an implantation-scheduled space in a chest is deformed due to capsular contracture to decrease its surface area. There is no probability that free movement of the breast becomes poor. Even though the construction is simple, a breast prosthesis having a good outer artificial coating can be provided.

### TECHNICAL FIELD

The present invention relates to a breast prosthesis including a core and an outer bag in which the core is contained in a movable state.

The present invention also relates to a method of breast augmentation to be applied to a human chest using a breast prosthesis including a core and an outer bag in which the core is contained in a movable state, including:

(a) a step of first partially cutting the human body to make an incised portion, and thereby forming an implantation-scheduled space in the chest for implanting the breast prosthesis;

(b) a step of then inserting the breast prosthesis into the implantation-scheduled space; and

(c) a step of then stitching up the incised portion.

### BACKGROUND ART

It is conventionally performed to reproduce a lost breast because the breast was lost as a result of an injury or a medical treatment, or enlarge breasts for the purpose of beauty. In this case, a breast prosthesis (so-called breast enlargement bag) is implanted under a pectoralis major muscle or mammary glands. As the breast prosthesis generally used is a silicone rubber bag filled with saline, silicone gel, or the like.

Breast prostheses generally used are roughly classified by texture into two kinds of a smooth type in which the outer surface of the bag is smooth; and a textured type in which the outer surface of the bag is textured with fine irregularities like ground glass. Such a breast prosthesis is provided with a silicone rubber tube detachably connected to the silicone rubber bag; and a valve to close a portion to which the tube was connected, for sealing the bag when the tube has been detached from the bag. In some breast prostheses, it is possible to control the filling amount of the filling material such as saline or silicone gel.

On the other hand, European Patent Application Laid-open No. 0 054 359 discloses a breast prosthesis of a double-layer structure. The breast prosthesis disclosed in this European Patent Application Laid-open No. 0 054 359 includes an outer bag made of an assimilatable material that disappears by being assimilated into the body's tissues when the breast prosthesis has been implanted into a woman chest; and an inner bag made of a non-assimilatable material that can continuously form an envelope of a core without being assimilated into the body's tissues. Therefore, in the case of the breast prosthesis disclosed in this European Patent Application Laid-open No. 0 054 359, a fibrous coating as will be described later is formed at a portion (that is, an outer surface of the outer bag) distant substantially by a certain distance from the core (that is, an inner portion made up of the inner bag and a filling material filling up the inner bag).
Patent Literature 1: European Patent Application Laid-open No. 0 054 359

In general, when a breast prosthesis is implanted into a human body, a fibrous coating is formed on the outer surface of the implanted breast prosthesis. The formation of such a fibrous coating is based on a natural human reaction for protecting the human body from foreign substances in the human body. In the case of a smooth type breast prosthesis as described above, because its outer surface is smooth, relatively slipping between the outer surface of the breast prosthesis and the inner surface of the fibrous coating formed on the outer surface of the breast prosthesis is apt to occur at the contact portion between them. Therefore, this smooth type breast prosthesis is deformed so that its surface area decreases as the thickness of the fibrous coating increases. This deformation phenomenon is called coating contracture or capsular contracture. That is, in the case of the above-described smooth type breast prosthesis, because the filling material filling up the bag is a fluid or a gel material, the volume of the breast prosthesis does not decrease even when the surface area of the breast prosthesis decreases as described above. Thus, the breast prosthesis has a problem that natural softness and natural movement of breast nature are lost from the breast prosthesis because its free shape change is suppressed to some extent with a decrease in the surface area (for example, the breast prosthesis becomes substantially spherical when the surface area is the minimum). In addition, the breast prosthesis also has a problem that the breast prosthesis is extremely deformed by such capsular contracture and may be broken. There is a possibility that the above capsular contracture in a smooth type breast prosthesis as described above may be avoided by frequently massaging almost daily the chest in which the breast prosthesis has been implanted. However, such massage has a problem of being accompanied by pain in many cases.

On the other hand, in the case of the above-described textured type breast prosthesis, fine irregularities like ground glass are formed on the outer surface. Therefore, in this breast prosthesis, its surface area is larger than that of a smooth type breast prosthesis of the same size (that is, volume), and a fibrous coating is formed along the irregularities on the outer surface of the breast prosthesis. Thus, because the thickness of the fibrous coating formed on the outer surface of such a breast prosthesis is hard to increase, capsular contracture as in the case of the above-described smooth type breast prosthesis is hard to occur. However, in the case of the textured type breast prosthesis, fine irregularities formed on the outer surface produce Velcro effect (in other words, fastening effect like a hook-and-loop fastener such as Velcro fastening (trademark)). Therefore, the bag of the breast prosthesis is united with the body's tissues. Thus, in the case of the textured type breast prosthesis, because natural movement of the breast prosthesis in the implanted portion of the chest is poor, there is a problem that the movement of the breast following a body action or the movement of the chest influenced by gravity appears odd.

Further, in the case of the breast prosthesis disclosed in the above European Patent Application Laid-open No. 0 054 359, a fibrous coating is formed at a portion (that is, the outer surface of the outer bag) distant substantially by a certain distance from the core (that is, the inner portion). Therefore, in the case of the breast prosthesis disclosed in this European Patent Application Laid-open No. 0 054 359, even though the outer surface of the outer bag is smooth, it is thinkable that there is an extremely low possibility that capsular contraction occurs that will occur in the case of the above-described smooth type breast prosthesis. Thus, the breast prosthesis disclosed in this European Patent Application Laid-open No. 0 054 359 may be superior on the point of keeping the shape of the breast prosthesis itself. However, on the point of reproducing the natural softness and the natural movement of a breast, it is thinkable that the breast prosthesis disclosed in this European Patent Application Laid-open No. 0 054 359 has the same problem as the above-described textured type breast prosthesis.

The present invention has been developed by focusing attention on problems as described above and to remove the problems.

### DISCLOSURE OF THE INVENTION

According to a first aspect of the present invention, a breast prosthesis including a core and an outer bag in which the core is contained in a movable state, is characterized in that the outer bag is made of a non-assimilatable material to function as an outer artificial coating; the outer bag has a large number of convexes and/or a large number of concaves formed integrally with a sheet-shaped base of the outer bag and disposed on an outer surface of the sheet-shaped base 13; and the convexes and/or the concaves are constructed so as to function as engaging means for a fibrous coating to be formed on an outer surface of the outer bag.

According to a second aspect of the present invention, a method of breast augmentation to be applied to a chest of a human body using a breast prosthesis including a core and an outer bag in which the core is contained in a movable state, including:

(a) a step of first partially cutting the human body to make an incised portion, and thereby forming an implantation-scheduled space in the chest for implanting the breast prosthesis;

(b) a step of then inserting the breast prosthesis into the implantation-scheduled space; and

(c) a step of then stitching up the incised portion, is characterized by using the breast prosthesis in which:

(d) the outer bag is made of a non-assimilatable material to function as an outer artificial coating;

(e) the outer bag has a large number of convexes and/or a large number of concaves formed integrally with a sheet-shaped base of the outer bag and disposed on an outer surface of the sheet-shaped base; and

(f) the convexes and/or the concaves function as engaging means for a fibrous coating to be formed on an outer surface of the outer bag.

According to the first and second aspects of the present invention, because a fibrous coating formed on the outer surface of the breast prosthesis (in other words, the outer surface of the outer bag to function as an outer artificial coating) is well connected with the outer surface of the outer bag to integrate both, the thickness of the fibrous coating is hard to increase. Therefore, because capsular contracture of the fibrous coating is hard to occur, there is no probability that the breast prosthesis is deformed to decrease its surface area. In addition, because the core is contained in the outer bag in a state that the core can move in the outer bag, the core can relatively freely move in the outer bag. Thus, there is no probability that free movement of the breast is lost. Further, because a large number of convexes and/or concaves that function as engaging means for the fibrous coating formed on the outer surface of the outer bag that functions as an outer artificial coating, are formed integrally with the sheet-shaped base, the structure of the breast prosthesis is simple.

In the first and second aspects of the present invention, it is preferable that the core includes an inner bag and a core filling material filling up the inner bag. This feature can simplify the construction of the core that gives natural softness to the breast.

In addition, in the first and second aspects of the present invention, it is preferable that each of the convexes and/or the concaves is a projection having a retaining portion. In this case, it is further preferable that the projection includes a columnar portion formed on the outer surface of the outer bag to protrude, and the retaining portion formed continuously from an end of the columnar portion. In this feature, because the fibrous coating is better connected with the outer surface of the outer bag, both can be fully integrated. In addition, the projection can be a single-pole type. Further, the projection can also be an arch type. The convexes and/or the concaves can also be a textured type that makes the outer surface of the sheet-shaped base textured with fine irregularities.

Further, in the first and second aspects of the present invention, it is preferable that a tube is detachably attachable to the outer bag so as to connect with the interior of the outer bag. In this feature, injection and discharge of the outer filling material into and from the outer bag can be surely performed by a simple operation, and in addition, the construction of the breast prosthesis can be further simplified.

In addition, in the second aspect of the present invention, the method can further include, between the step (b) and the step (c):

(g) a step of injecting an outer filling material into the outer bag through a tube. In addition, the method can further include, between the step (g) and the step (c):

(h) a step of discharging an adequate amount of outer filling material from the outer bag to the outside to adjust the shape of the breast of the human body. In addition, the method can further include, between the step (h) and the step (c):

(i) a step of detaching the tube from the outer bag and then closing the tube connection portion of the outer bag with a valve.

### BRIEF DESCRIPTION OF DRAWINGS

[FIG. 1]

FIG. 1 is a schematic vertical sectional view of a chest showing an implantation-scheduled portion formed in the chest for implanting a breast prosthesis according to an embodiment to which the present invention is applied, into the chest of a human body such as a woman;

[FIG. 2]

FIG. 2 is a vertical sectional view like FIG. 1, showing a state immediately after the breast prosthesis is implanted into the implantation-scheduled portion of FIG. 1;

[FIG. 3]

FIG. 3 is a vertical sectional view like FIG. 2, showing a state immediately after saline is injected into an outer bag of the breast prosthesis of FIG. 2;

[FIG. 4]

FIG. 4 is a vertical sectional view like FIG. 2, showing a state immediately after a desired amount of saline is discharged from the breast prosthesis of FIG. 3;

[FIG. 5]

FIG. 5 is a vertical sectional view like FIG. 2, showing a state immediately after a tube is removed from the breast prosthesis of FIG. 4;

[FIG. 6]

FIG. 6 is a partially vertically sectional enlarged partial view showing a first specific example of an outer bag of the breast prosthesis shown in FIG. 2; and

[FIG. 7]

FIG. 7 is an enlarged view like FIG. 6, showing a second specific example of an outer bag of the breast prosthesis shown in FIG. 2.

### BEST FORM FOR CARRYING OUT THE INVENTION

Next, an embodiment to which the present invention is applied will be described with reference to drawings by dividing into items of “1. General Construction of Breast Prosthesis”, “2. Shape of Outer Surface of Outer Bag”, and “3. Procedure of Breast Augmentation”.

### 1. General Construction of Breast Prosthesis

As shown in FIG. 2, a breast prosthesis 1 may include:

(a) an inner bag 3 having a smooth slippery outer surface, and forming an envelope of a core 2;

(b) an outer bag 4 having a smooth slippery inner surface, containing therein the core 2 in a freely movable state, and functioning as an outer artificial capsule;

(c) a tube 5 detachably connected to the outer bag 4; and

(d) a valve (not shown) to close a portion to which the tube 5 was connected, for sealing the outer bag 4 when the tube 5 has been detached from the outer bag 4. The inner bag 3 may be beforehand filled with a core filling material 6 of a gel material such as silicone gel or a compatible solution such as saline to form the core 2 made up of the inner bag 3 and the core filling material 6 filling up the inner bag 3. In the outer bag 4, between the outer surface of the inner bag 3 and the inner surface of the outer bag 4, all of an outer filling material 7 of a compatible solution such as saline, or the like, may have been discharged. However, in accordance with convenience of breast augmentation, the outer bag 4 may have been beforehand filled with some amount of outer filling material.

As shown in FIG. 2, each of the inner bag 3 and the outer bag 4 is made of a non-assimilatable material (that is, a material substantially not to be assimilated by the body's tissues) that can continuously (preferably permanently) exist as an envelope of the core 2 and an envelope of the outer portion without being assimilated in the body's tissues when the breast prosthesis 1 is implanted into a chest 12 of a human body 11 such as a woman; and can be formed by shaping into a sheet and then into a bag. Such a non-assimilatable material may be a material having plasticity or flexibility, such as silicone rubber or foamed PTFE (that is, foamed polytetrafluoroethylene). The tube 5 can be used for discharging gas such as air and the outer filling material 7 in the outer bag 4 to the outside when need, or injecting the outer filling material 7 into the outer bag 4 from the outside. The tube 5 may be made of a non-assimilatable material such as silicone rubber or foamed PTFE, or another appropriate material as needed. Further, each of the tube 5 and the valve may be known one that is conventionally used for a bag of a breast prosthesis.

### 2. Shape of Outer Surface of Outer Bag

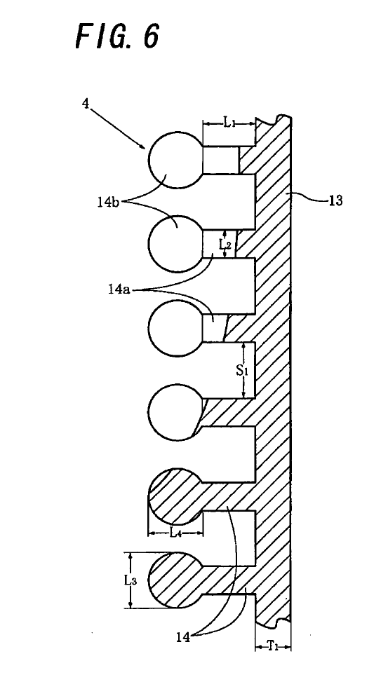

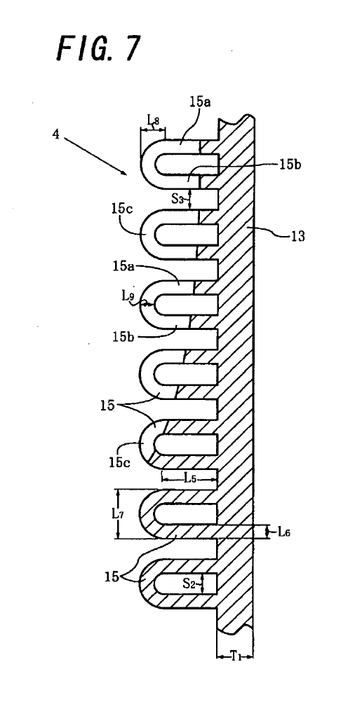

On the outer surface of the outer bag 4, as shown in FIGS. 6 and 7, there may be provided a large number of projections 14 or 15 formed integrally with a sheet-shaped base 13 of the outer bag 4 by molding or the like. The projections 14 or 15 function as engaging means for engaging with a fibrous coating formed on the outer surface of the outer bag 4. The projections 14 or 15 may be formed on substantially the whole area of the outer surface of the sheet-shaped base 13. The projections 14 or 15 can be formed integrally with the sheet-shaped base 13 in the manner that both (that is, the projections 14 or 15 and the sheet-shaped base 13 are molded at once from the same molding material as the sheet-shaped base 13. Further, it is not indispensable that the projections 14 or 15 are evenly formed on substantially the whole area of the outer surface of the sheet-shaped base 13. From the viewpoint of practicality, in general, the projections 14 or 15 are formed preferably in a region of 50 to 100%, more preferably in a region of 70 to 100%, furthermore preferably in a region of 90 to 100% of the area of the outer surface of the sheet-shaped base 13. Therefore, in the present invention, a large number of projections 14 or 15 may be arranged on the outer surface of the outer bag 4 so as to make a substantially stripe shape, a substantially lattice shape, a substantially grid shape, a substantially polka-dot shape, or another shape on the whole. In other words, a large number of projections 14 or 15 may make groups each constituted by a plurality of projections, on a plurality of regions of the sheet-shaped base 13.

FIG. 6 shows a first specific example of projections 14 or 15 formed on the outer surface of the outer bag 4. In the first specific example shown in FIG. 6, each of the projections 14 is a single-pole type. Therefore, each projection 14 may be made up of one columnar portion 14a of a substantially columnar shape extending outward from the outer surface of the sheet-shaped base 13 so as to be substantially perpendicular to the outer surface; and one retaining portion 14b of a head shape formed integrally with the columnar portion 14a by molding both at once so as to be continuously connected to the end of the columnar portion 14a and to be somewhat larger than the columnar portion 14a in a direction substantially parallel to the outer surface of the sheet-shaped base 13. In this case, the columnar portion 14a constitutes a constricted portion of the projection 14, and a lower half portion (that is, a base portion) of the retaining portion 14b also constitutes the constricted portion of the projection 14.

FIG. 7 shows a second specific example of projections 14 or 15 formed on the outer surface of the outer bag 4. In the second specific example shown in FIG. 7, each of the projections 15 is a double-pole type or an arch type. Therefore, each projection 15 may be made up of two columnar portions 15a and 15b of a substantially columnar shape extending outward from the outer surface of the sheet-shaped base 13 so as to be substantially perpendicular to the outer surface; and one retaining portion 15c as an interconnecting portion formed integrally with the two columnar portions 15a and 15b by molding both at once so as to interconnect the ends of the two columnar portions 15a and 15b by forming both ends continuously to the ends of the two columnar portions 15a and 15b. The retaining portion 14b or 15c functions as retaining means for a fibrous coating formed on the outer surface of the outer bag 4.

In the first and second specific examples shown in FIGS. 6 and 7, the thickness T₁ of the sheet-shaped base 13 is about 0.5 mm. In the first specific examples shown in FIG. 6, the length L₁ of each columnar portion 14a is about 0.75 mm. The maximum length L₂ of each columnar portion 14a substantially parallel to the outer surface of the sheet-shaped base 13 (in the case of a substantially cylindrical column shape, the diameter in a cross section) is about 0.4 mm. The maximum lengths L₃ and L₄ of each retaining portion 14b substantially parallel and substantially perpendicular to the outer surface of the sheet-shaped base 13 (in the case of a substantially spherical shape, its diameter) are about 0.8 mm. The interval S₁ between each pair of neighboring columnar portions 14a is about 0.8 mm.

In the second specific example shown in FIG. 7, the length L₅ of each of columnar portions 15a and 15b is about 0.75 mm. The maximum length L₆ of each of columnar portions 15a and 15b substantially parallel to the outer surface of the sheet-shaped base 13 (in the case of a substantially cylindrical column shape, the diameter in a cross section) is about 0.2 mm. The maximum length L₇ of each retaining portion 15c substantially parallel to the outer surface of the sheet-shaped base 13 is about 0.7 mm. The maximum length L₈ of each retaining portion 15c substantially perpendicular to the outer surface of the sheet-shaped base 13 is about 0.35 mm. The maximum length L₉ of each retaining portion 15c substantially perpendicular to a longitudinal axis of the retaining portion 15c (in the case of a circular cross section, its diameter) is about 0.2 mm. The interval S₂ between a pair of columnar portions 15a and 15b of each projection 15 is about 0.3 mm. The interval S₃ between a pair of neighboring columnar portions 15a and 15b of each pair of neighboring projections 15 is about 0.3 mm.

Therefore, in the present invention, from the viewpoint of practicality, in general, one, some, or all of the numerical ranges described in the following items (a) to (n) are preferably satisfied. The numerical ranges in the parentheses described in the following items (a) to (n) are numerical ranges to be more preferably satisfied.

(a) The thickness T₁ of the sheet-shaped base 13: a range of 0.1 to 2.5 mm (0.2 to 1.2 mm);

(b) The lengths L₁ and L₅ of the columnar portions 14a, 15a, and 15b: a range of 0.15 to 4 mm (0.3 to 2 mm);

(c) The maximum lengths L₂ and L₆ of the columnar portions 14a, 15a, and 15b substantially parallel to the outer surface of the sheet-shaped base 13: a range of 0.05 to 2 mm (0.1 to 1 mm);

(d) The maximum lengths L₃, L₄, L₇, and L₈ of the retaining portions 14b and 14c substantially parallel and/or substantially perpendicular to the outer surface of the sheet-shaped base 13: a range of 0.1 to 3 mm (0.2 to 1.5 mm);

(e) The interval S₁ between each pair of neighboring columnar portions 14a of the single-pole type projections 14: a range of 0.2 to 4 mm (0.4 to 2 mm);

(f) The maximum length L₉ of the retaining portion 15c of each arch type projection 15 substantially perpendicular to a longitudinal axis of the retaining portion 15c: a range of 0.05 to 2 mm (0.1mm to 1 mm);

(g) The interval S₂ between a pair of columnar portions 15a and 15b of each arch type projection 15: a range of 0.075 to 3 mm (0.15 to 2 mm);

(h) The interval S₃ between a pair of neighboring columnar portions 15a and 15b of each pair of neighboring arch type projections 15: a range of 0.075 to 3 mm (0.15 to 2 mm);

(i) The ratios of the lengths L₁ and L₂ described in the above item (b) to the thickness T₁ described in the above item (a): a range of 0.3 to 6 (0.6 to 3);

(j) The ratios of the maximum lengths L₂ and L₆ described in the above item (c) to the thickness T₁ described in the above item (a): a range of 0.1 to 4 (0.3 to 2);

(k) The ratios of the maximum lengths L₃, L₄, L₇, and L₈ described in the above item (d) to the thickness T₁ described in the above item (a): a range of 0.2 to 6 (0.4 to 3);

(l) The ratios of the maximum lengths L₂ and L₆ described in the above item (c) to the lengths L₁ and L₅ described in the above item (b); a range of 0.05 to 4 (0.1 to 2);

(m) The ratios of the maximum lengths L₃, L₄, L₇, and L₈ described in the above item (d) to the lengths L₁ and L₅ described in the above item (b); a range of 0.1 to 5 (0.2 to 3); and

(n) The ratios of the maximum lengths L₃, L₄, L₇, and L₈ described in the above item (d) to the maximum lengths L₂ and L₆ described in the above item (c): a range of 0.2 to 10 (0.4 to 6).

In the first and second specific examples shown in FIGS. 6 and 7, each of the columnar portions 14a, 15a, and 15b has a substantially cylindrical column shape. Each retaining portion 14b is substantially spherical. Each retaining portion 15c is substantially semi-ring-shaped as a whole, and its cross section is substantially circular. A cross section of the retaining portion 15c has substantially the same shape as a cross section of each of the columnar portions 15a and 15c, and they are continuously formed so that their interconnection portion is smooth each other. In the present invention, however, each of the columnar portions 14a, 15a, and 15b may have an arbitral substantially columnar shape as a whole, such as a substantially quadrangular prism shape, a substantially hexagonal column shape, a substantially elliptic column shape, or a substantially circular truncated cone shape. In addition, each retaining portion 14b may have an arbitral block shape as a whole, such as a substantially semispherical shape, a substantially ellipsoidal shape, a flattened substantially cylindrical column shape, a flattened substantially elliptic column shape, a substantially rectangular parallelepiped shape, a substantially cubic shape, or a substantially hexagonal column shape. Further, each retaining portion 15c may have an arbitral shape as a whole, such as a substantially straight rod shape. In addition, a cross section of each retaining portion 15c may have an arbitral shape such as a substantially elliptic shape, a substantially elongated circular shape, a substantially rectangular shape, a substantially square shape, or a substantially hexagonal shape.

In the first and second specific examples shown in FIGS. 6 and 7, each of the projections 14 and 15 is made up of a columnar portion 14a or 15a and a retaining portion 14b or 15c. However, for example, in the case of each projection 14 shown in FIG. 6, a retaining portion (in other words, a protruding integrated portion) 14b can be integrally formed directly on the outer surface of the sheet-shaped base 13 of the outer bag 4 with omitting a columnar portion 14a.

Further, in a third specific example (not shown) of the present invention, the outer surface itself of the sheet-shaped base 13 can be formed into a ground glass shape (in other words, textured type) textured with fine irregularities like ground glass. Such a textured type concavo-convex surface can form a concavo-convex integrated portion constituted by the outer surface of the outer bag 4 that functions as an outer artificial coating. Such a textured type concavo-convex surface can be constituted by the outer surface of the sheet-shaped base 13 itself when or after the sheet-shaped base 13 is formed. When the sheet-shaped base 13 is formed, the concavo-convex surface can be formed by molding. After the sheet-shaped base 13 is formed, the concavo-convex surface can be formed by sand blast or the like.

From the viewpoint of practicality, in general, the height and/or the depth of fine convexes and/or fine concaves on the textured type concavo-convex surface is preferably within a range of 0.01 to 1 mm, more preferably within a range of 0.05 to 0.5 mm. From the viewpoint of practicality, in general, the maximum length of each of the convexes and/or the concaves substantially parallel to the outer surface of the sheet-shaped base 13 (in the case of circular convex and/or concave, its diameter) is preferably within a range of 0.01 to 3 mm, more preferably within a range of 0.05 to 1.5 mm. Further, a large number of convexes and/or concaves can be formed wholly or partially on the outer surface of the sheet-shaped base 13, like the projections 14 or 15 shown in FIGS. 6 and 7. The number of convexes and/or concaves per 1 cm on the outer surface of the sheet-shaped base 13 (the number of convexes when only the convexes exist, the number of concaves when only the concaves exist, and the total number of convexes and concaves when both exist) may be, for example, forty. From the viewpoint of practicality, in general, the number of convexes and concaves per 1 cm on the outer surface of the sheet-shaped base 13 is preferably 4 to 400, more preferably 8 to 200, furthermore preferably 20 to 100.

### 3. Procedure of Breast Augmentation

A specific example of the procedure of applying breast augmentation to a chest 12 of a human body 11 such as a woman using a breast prosthesis 1 shown in FIGS. 2, 6 and 7 may be as described in the following items (a) to (e).

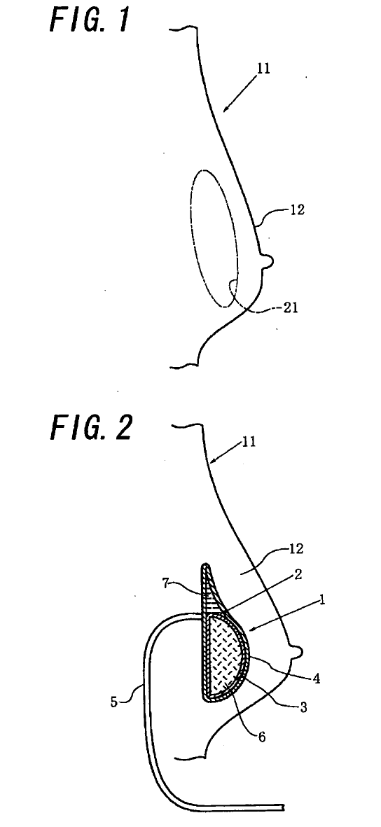

(a) First, as shown in FIG. 1, a space 21 for implanting the breast prosthesis shown in FIG. 2 is formed in a chest 12 of a human body 11 such as a woman. Such an implantation-scheduled space 21 may be formed either under mammary glands or under a pectoralis major muscle, which is determined in consideration of the conditions of the breast, the size of the breast prosthesis 1, and so on. The implantation-scheduled space 21 is ensured by inserting a peeling member (not shown) either under the mammary glands or under the pectoralis major muscle to partially peel the mammary glands or partially peel the pectoralis major muscle together with the mammary glands. The peeling member can be inserted through an incised portion obtained by incising an armpit of the human body 11 or a lower portion of the breast.

(b) Next, as shown in FIG. 2, the breast prosthesis 1 is inserted into the implantation-scheduled space 21 through the portion through which the peeling member was inserted. At this time, the breast prosthesis 1 is preferably inserted into the implantation-scheduled space 21 in a state that the outer bag 4 is filled with no or a small amount of outer filling material 7 such as saline. In this inserting method, the breast prosthesis 1 can be inserted even when the incised portion of the human body 11 is small. Thus, this method is particularly advantageous in breast augmentation of a type in which an armpit is incised to insert the breast prosthesis 1 as described above.

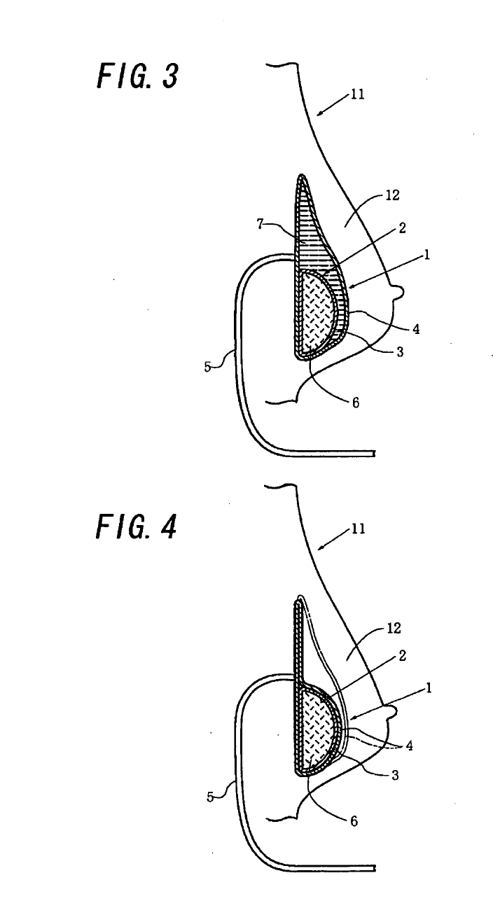

(c) Next, as shown in FIG. 3, an outer filling material 7 such as saline is injected into the outer bag 4 of the breast prosthesis 1 through the tube 5 to expand the outer bag 4. Thus, the outer bag 4 fully occupies the implantation-scheduled space 21 shown in FIG. 1 without wrinkles or folds.

(d) Next, as shown in FIG. 4, for adjusting the shape of the breast of the human body 11, an adequate amount (in general, almost all) of outer filling material 7 such as saline is discharged from the outer bag 4 to the outside through the tube 5. In FIG. 4, the outer bag 4 shown by chain lines indicates a state shown by solid lines in FIG. 3. While an adequate amount (in general, almost all) of outer filling material 7 such as saline is discharged from the outer bag 4 to the outside by a syringe pump (not shown), the position of the breast prosthesis 1 to the chest 12 of the human body 11 is adjusted to adjust the shape of the breast of the human body 11. In this case, although the volume of the outer bag 4 decreases, a state that the inner surface of the implantation-scheduled space 21 and the outer surface of the outer bag 4 are in close contact with each other can be kept by pressing the breast of the human body 11 from the outside even after the breast augmentation. This can suppress formation of a hematoma, and promote the integration of the body's tissues and the outer bag 4.

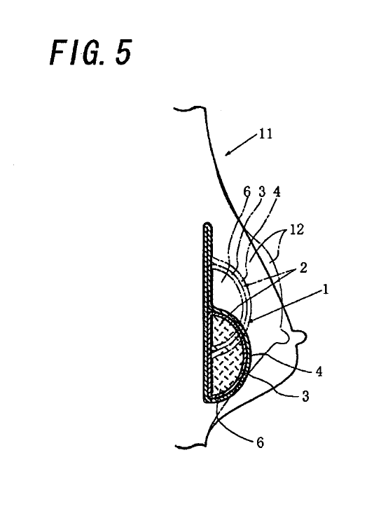

(e) Next, as shown in FIG. 5, the tube 5 is detached from the outer bag 4 of the breast prosthesis 1. The tube connection portion of the outer bag 4 is then closed with a valve (not shown) to seal the outer bag 4. Next, the incised portion of the human body 11 incised as described in the item (a) is stitched up by an adequate method as well known.

In the breast prosthesis 1 having been implanted into the implantation-scheduled space 21 in the chest 12 of the human body 11 to which the breast augmentation was applied as described above, a large number of projections 14 shown in FIG. 6, a large number of projections 15 shown in FIG. 7, or a large number of convexes and/or a large number of concaves of the above-described third specific example are formed on the outer surface of the outer bag 4. Therefore, a fibrous coating formed on the outer surface of the outer bag 4 due to change with time is well connected with the outer surface of the outer bag 4 by mutual action of the columnar portions 14a or 15a and 15b and the retaining portions 14b or 15c or the above-described Velcro effect by action of the above-described convexes and/or concaves, and thereby both are integrated. Thus, because the thickness of the fibrous coating does not increase to decrease the surface area of the outer bag 4, there is almost no probability of occurrence of capsular contracture of the fibrous coating. Therefore, the natural softness of the breast of the human body 11 is almost never inhibited. In addition, because the fibrous coating is integrally connected with the outer surface of the outer bag 4 as described above, the movement of the outer bag 4 near the breast of the human body 11 is restricted to some extent. However, because the inner bag 3 that is contained in the outer bag 4 in a freely movable state can freely move in the outer bag 4, the natural movement of the breast of the human body 11 is not particularly inhibited. The inner bag 3 shown by chain lines in FIG. 5 indicates a state that the inner bag 3 has moved substantially upward in the outer bag 4 from the state shown by solid lines in FIG. 5.

Although the specific preferred embodiments of the present invention have been described with reference to the accompanying drawings, it should be understood that the present invention is never limited to the detailed embodiments and various changes and corrections may be made there by those skilled in the art without departing from the scope and spirit of the invention defined by the appended claims.

For example, in the above embodiments, as projections formed on the outer surface of the outer bag 4, the single-pole type projections 14 or the double-pole type projections 15 are used. However, single-pole type projections 14 and double-pole type projections 15 can be formed together on the outer surface of the common outer bag 4. In addition, as the above projections, convexes and/or concaves of arbitrary shapes other than the single-pole type projections 14 and the double-pole type projections 15 (for example, the convexes and/or concaves of the above-described third specific example) can be used.

In addition, in the above embodiments, the retaining portions 14b or 15c are provided on all of a large number of projections 14 or 15. However, retaining portions 14b or 15c may be provided on only some of a large number of projections 14 or 15, and no retaining portions 14b or 15c may be provided on the remaining projections. Further, the retaining portions 14b or 15c can be omitted from all of a large number of projections 14 or 15 so that all of a large number of projections 14 or 15 function as merely engaging means without retaining function.

Further, in the above embodiments, the columnar portions 14a or 15a and 15b of the projections 14 or 15 of the outer bag 4 extend substantially perpendicularly to the outer surface of the sheet-shaped base 13. However, it is not indispensable that the columnar portions 14a or 15a and 15b extend substantially perpendicularly to the outer surface of the sheet-shaped base 13. They may extend somewhat obliquely to the outer surface of the sheet-shaped base 13.

1. A breast prosthesis comprising a core (2) and an outer bag (4) in which the core (2) is contained in a movable state, characterized in that
the outer bag (4) is made of a non-assimilatable material to function as an outer artificial coating;
the outer bag (4) comprises a large number of convexes (14, 15) and/or a large number of concaves formed integrally with a sheet-shaped base (13) of the outer bag (4) and disposed on an outer surface of the sheet-shaped base (13); and
the convexes (14, 15) and/or the concaves are constructed so as to function as engaging means for a fibrous coating to be formed on an outer surface of the outer bag (4).

2. The breast prosthesis according to claim 1, characterized in that the core (2) comprises an inner bag (3) and a core filling material (6) filling up the inner bag (3).

3. The breast prosthesis according to claim 2, characterized in that the core filling material (6) is a gel material and/or a compatible solution.

4. The breast prosthesis according to any of claims 1 to 3, characterized in that the space between an outer surface of the core (2) and an inner surface of the outer bag (4) is filled up with an outer filling material (7).

5. The breast prosthesis according to claim 4, characterized in that the outer filling material (7) is a gel material and/or a compatible solution.

6. The breast prosthesis according to any of claims 1 to 5, characterized in that each of the convexes and/or the concaves is a projection (14, 15) comprising a retaining portion (14b, 15c).

7. The breast prosthesis according to any of claims 1 to 5, characterized in that each of the convexes and/or the concaves is a projection (14) comprising a constricted portion.

8. The breast prosthesis according to claim 6, characterized in that the projection (14, 15) comprises a columnar portion (14a, 15a, 15b) formed on the outer surface of the outer bag (4) to protrude, and the retaining portion (14b, 15c) formed continuously from an end of the columnar portion (14a, 15a, 15b).

9. The breast prosthesis according to claim 8, characterized in that the projection (14, 15) is a single-pole type projection (14).

10. The breast prosthesis according to claim 8, characterized in that the projection (14, 15) is an arch type projection (15).

11. The breast prosthesis according to any of claims 1 to 5, characterized in that the convexes and/or the concaves are textured type convexes and/or concaves that make the outer surface of the sheet-shaped base (13) textured with fine irregularities.

12. The breast prosthesis according to any of claims 1 to 11, characterized in that a tube (5) is detachably attachable to the outer bag (4) so as to connect with the interior of the outer bag (4).

13. The breast prosthesis according to claim 12, characterized in that a valve is provided for the outer bag (4) to close a portion of the outer bag (4) to which the tube (5) was connected, for sealing the outer bag (4) when the tube (5) has been detached from the outer bag (4).

14. The breast prosthesis according to any of claims 1 to 13, characterized in that the convexes (14, 15) and/or the concaves are formed within a range of 50 to 100%, preferably 70 to 100%, more preferably 90 to 100% of the area of the outer surface of the sheet-shaped base (13).

15. The breast prosthesis according to any of claims 1 to 14, characterized in that a thickness (T₁) of the sheet-shaped base (13) is within a range of 0.1 to 2.5 mm, preferably 0.2 to 1.2 mm.

16. The breast prosthesis according to any of claims 8 to 10, characterized in that a length (L₁, L₂) of each columnar portion (14a, 15a, 15b) is within a range of 0.15 to 4 mm, preferably 0.3 to 2 mm.

17. The breast prosthesis according to any of claims 8 to 10 and 16, characterized in that the maximum length (L₂, L₆) of each columnar portion (14a, 15a, 15b) substantially parallel to the outer surface of the sheet-shaped base (13) is within a range of 0.05 to 2 mm, preferably 0.1 to 1 mm.

18. The breast prosthesis according to any of claims 6, 8 to 10, 16, and 17, characterized in that the maximum length (L₃, L₄, L₇, L₈) of each retaining portion (14b, 15c) substantially parallel and/or substantially perpendicular to the outer surface of the sheet-shaped base (13) is within a range of 0.1 to 3 mm, preferably 0.2 to 1.5 mm.

19. The breast prosthesis according to claim 9, characterized in that the interval (S₁) between a pair of neighboring columnar portions (14a) of the single-pole type projections (14) is within a range of 0.2 to 4 mm, preferably 0.4 to 2 mm.

20. The breast prosthesis according to claim 10, characterized in that the maximum length (L₉) of each retaining portion (14b, 15c)(15c) of the arch type projection (14, 15)(15) substantially perpendicular to a longitudinal axis of the retaining portion is within a range of 0.05 to 2 mm, preferably 0.1 to 1 mm.

21. The breast prosthesis according to claim 10 or 20, characterized in that the interval (S₂) between a pair of columnar portions (15a, 15b) of each arch type projection (15) is within a range of 0.075 to 3 mm, preferably 0.15 to 2 mm.

22. The breast prosthesis according to any of claims 10, 20, and 21, characterized in that the interval (S₃) between a pair of neighboring columnar portions (14a, 15a, 15b)(15a, 15b) of a pair of neighboring arch type projections (14, 15)(15) is within a range of 0.075 to 3 mm, preferably 0.15 to 2 mm.

23. The breast prosthesis according to any of claims 8 to 10, 16, 17, and 19 to 22, characterized in that the ratio of the length (L₁, L₅) of each columnar portion (14a, 15a, 15b) to the thickness (T₁) of the sheet-shaped base (13) is within a range of 0.3 to 6, preferably 0.6 to 3.

24. The breast prosthesis according to any of claims 8 to 10, 16, 17, and 19 to 23, characterized in that the ratio of the maximum length (L₂, L₆) of each columnar portion (14a, 15a, 15b) substantially parallel to the outer surface of the sheet-shaped base (13) to the thickness (T₁) of the sheet-shaped base (13) is within a range of 0.1 to 4, preferably 0.3 to 2.

25. The breast prosthesis according to any of claims 6, 8 to 10, and 16 to 24, characterized in that the ratio of the maximum length (L₃, L₄, L₇, L₈) of each retaining portion (14b, 15c) substantially parallel and/or substantially perpendicular to the outer surface of the sheet-shaped base (13) to the thickness (T₁) of the sheet-shaped base (13) is within a range of 0.2 to 6, preferably 0.4 to 3.

26. The breast prosthesis according to any of claims 7 to 10, 16, 17, and 19 to 25, characterized in that the ratio of the maximum length (L₂, L₆) of each columnar portion (14a, 15a, 15b) substantially parallel to the outer surface of the sheet-shaped base (13) to the length (L₅) of each columnar portion (14a, 15a, 15b) is within a range of 0.05 to 4, preferably 0.1 to 2.

27. The breast prosthesis according to any of claims 8 to 10, 16, 17, and 19 to 26, characterized in that the ratio of the maximum length (L₃, L₄, L₇, L₈) of each retaining portion (14b, 15c) substantially parallel and/or substantially perpendicular to the outer surface of the sheet-shaped base (13) to the length (L₁, L₅) of each columnar portion (14a, 15a, 15b) is within a range of 0.1 to 5, preferably 0.2 to 3.

28. The breast prosthesis according to any of claims 8 to 10, 16, 17, and 19 to 27, characterized in that the ratio of the maximum length (L₃, L₄, L₇, L₈) of each retaining portion (14b, 15c) substantially parallel and/or substantially perpendicular to the outer surface of the sheet-shaped base (13) to the maximum length (L₂, L₆) of each columnar portion (14a, 15a, 15b) substantially parallel to the outer surface of the sheet-shaped base (13) is within a range of 0.2 to 10, preferably 0.4 to 6.

29. The breast prosthesis according to any of claims 8 to 10, 16, 17, and 19 to 28, characterized in that each columnar portion (14a, 15a, 15b) has a substantially cylindrical column shape.

30. The breast prosthesis according to claim 9 or 19, characterized in that each retaining portion (14b) is substantially spherical.

31. The breast prosthesis according to claim 10, 20, or 21, characterized in that each retaining portion (15c) is substantially semi-ring-shaped as a whole, and its cross section is substantially circular.

32. The breast prosthesis according to claim 11, characterized in that the height and/or depth of each of the textured type convexes and/or concaves is within a range of 0.01 to 1 mm, preferably 0.05 to 0.5 mm.

33. The breast prosthesis according to claim 11 or 32, characterized in that the maximum length of each of the textured type convexes and/or concaves is within a range of 0.01 to 3 mm, preferably 0.05 to 1.5 mm.

34. The breast prosthesis according to any of claims 11, 32 and 33, characterized in that the number of textured type convexes and/or concaves per 1 cm is within a range of 4 to 400, preferably 8 to 200, more preferably 20 to 100.

35. The breast prosthesis according to claim 8, characterized in that single-pole type projections (14) and double-pole type projections (15) are provided together.

36. The breast prosthesis according to claim 8, characterized in that projections (14, 15) each having a retaining portion (14b, 15c) and projections each having no retaining portion are provided together as the projections.

37. The breast prosthesis according to any of claims 1 to 5, characterized in that the convexes and/or the concaves are projections each having no retaining portion (14b, 15c).

38. A method of breast augmentation to be applied to a chest (12) of a human body (11) using a breast prosthesis (1) comprising a core (2) and an outer bag (4) in which the core (2) is contained in a movable state, comprising:
(a) a step of first partially cutting the human body (11) to make an incised portion, and thereby forming an implantation-scheduled space (21) in the chest (12) for implanting the breast prosthesis (1);
(b) a step of then inserting the breast prosthesis (1) into the implantation-scheduled space (21); and
(c) a step of then stitching up the incised portion, characterized by using the breast prosthesis (1) in which:
(d) the outer bag (4) is made of a non-assimilatable material to function as an outer artificial coating;
(e) the outer bag (4) comprises a large number of convexes (14, 15) and/or a large number of concaves formed integrally with a sheet-shaped base (13) of the outer bag (4) and disposed on an outer surface of the sheet-shaped base (13); and
(f) the convexes (14, 15) and/or the concaves function as engaging means for a fibrous coating to be formed on an outer surface of the outer bag (4).

39. The method of breast augmentation according to claim 38, characterized in that each of the convexes and/or the concaves is a projection (14, 15) comprising a retaining portion (14b, 15c).

40. The method of breast augmentation according to claim 38 or 39, characterized by further comprising, between the step (b) and the step (c):
(g) a step of injecting an outer filling material (7) into the outer bag (4) through a tube (5).

41. The method of breast augmentation according to any of claims 38 to 40, characterized by further comprising, between the step (g) and the step (c):
(h) a step of discharging an adequate amount of outer filling material (7) from the outer bag (4) to the outside to adjust the shape of the breast of the human body (11).

42. The method of breast augmentation according to any of claims 38 to 41, characterized by further comprising, between the step (h) and the step (c):
(i) a step of detaching the tube (5) from the outer bag (4) and then closing the tube connection portion of the outer bag (4) with a valve.
